(19) United States
(12) Patent Application Publication
(10) Pub. No.: US 2026/0018291 A1
(43) Pub. Date: Jan. 15, 2026

(54) Domain Adaptation Engine(s) For Cell-Free DNA Fragmentomics

(71) Applicant: The Regents of the University of Colorado, a body corporate, Denver, CO (US)

(72) Inventors: Natalie Rose Davidson, Aurora, CO (US); Srinivas Ramachandran, Denver, CO (US); Casey Stephen Greene, Denver, CO (US)

(21) Appl. No.: 19/268,182

(22) Filed: Jul. 14, 2025

Related U.S. Application Data
(60) Provisional application No. 63/671,407, filed on Jul. 15, 2024.

(51) Int. Cl. G16H 50/20; G16B 20/20; G16B 40/20; G16H 15/00; G16H 50/70

(52) U.S. Cl. CPC G16H 50/20; G16B 20/20; G16B 40/20; G16H 15/00; G16H 50/70

(57) ABSTRACT

Systems and methods for predicting tissue-of-origin for diseased tissues using cell-free DNA (cfDNA) are provided herein. In an aspect, a domain adaptation engine receives a cfDNA sample from a subject and generates cfDNA fragmentation data from the cfDNA sample. The domain adaptation engine deconvolutes the cfDNA fragmentation data using Assay for Transposase-Accessible Chromatin using sequencing (ATAC-Seq) data from multiple tissue and cell types to generate deconvoluted cfDNA fragmentation data. In an example, the domain adaptation engine deconvolutes the cfDNA fragmentation data using a machine learning (ML) system trained to translate between the ATAC-Seq data and cfDNA data. Subsequently, the domain adaptation engine detects a diseased tissue signature within the deconvoluted cfDNA fragmentation data and generates a tissue or cell-type-of-origin prediction for the diseased tissue signature based on the deconvoluted cfDNA fragmentation data.

### TECHNICAL FIELD

Aspects of the disclosure are related to the field of disease detection, in particular to technology for determining tissue-of-origin for diseased tissue using cell-free DNA (cfDNA).

### BACKGROUND

Accurately detecting and identifying the tissue-of-origin of diseased tissue is an important step for effective diagnosis, prognosis, and treatment planning across a wide range of medical conditions. Many diseases—such as cancer, autoimmune disorders, and organ transplant rejection—manifest through tissue-specific changes that influence clinical management. Knowing the exact tissue or cell type from which the disease originates enables clinicians to select targeted therapies, predict disease progression, and monitor treatment response with greater precision. For example, tumors arising from different cell types within the same tissue—or tumors with similar histological appearance but distinct gene expression profiles—may respond differently to chemotherapy or immunotherapy, despite appearing morphologically similar. Similarly, identifying which organ is undergoing immune attack in autoimmune disease or transplant rejection is informative for tailoring immunosuppressive regimens. Thus, resolving the tissue-of-origin is often an important step in implementing personalized, tissue-informed therapeutic strategies.

### SUMMARY

Systems and methods for detecting diseased tissue and predicting tissue-of-origin for the diseased tissue suing cfDNA are provided herein. In an aspect, a domain adaptation engine is provided that leverages Assay for Transposase-Accessible Chromatin using sequencing (ATAC-Seq) data from multiple tissue types to deconvolute cfDNA for disease detection. As will be described in greater detail below, a cfDNA sample may be received from a subject, such as a patient, and cfDNA fragmentation data may be generated from the cfDNA sample. The cfDNA fragmentation data may be deconvoluted by the domain adaptation engine to generate deconvoluted cfDNA fragmentation data. From the deconvoluted cfDNA fragmentation data, the domain adaptation engine may detect a diseased tissue signature and based on tissue-specific chromatin accessibility signatures expressed within the ATAC-Seq data, generate a tissue-of-origin prediction for the diseased tissue signature.

In some embodiments, to deconvolute the cfDNA fragmentation data, the domain adaptation engine leverages a machine-learning (ML) system containing multiple variational autoencoders (VAEs), each trained to predict a different variable source. For example, the ML system may include four VAEs directed to a technology specific variability source, a tissue type variability source, a blood cell-type proportion variability source, and a remaining variability source. Using the cell-type-specific ATAC-Seq data, the VAEs may be trained to encode the cfDNA fragmentation data into a latent space representation according to a respective variability source. Since the VAEs model each source of variability within the cfDNA fragmentation data, each latent space represents the cfDNA fragmentation data independent of one another, including a latent space that models cell-type or tissue-type-specific signals. The latent space representations from each VAE may then be concatenated to generate a combined latent space representation representing the deconvoluted cfDNA fragmentation data. Based on the deconvoluted cfDNA fragmentation data, the domain adaptation engine may detect whether any diseased tissue or cell-type signatures are present and, if so, whether the diseased tissue signature maps to any known tissue-specific chromatin accessibility signatures.

This Summary is provided to introduce a selection of concepts in a simplified form that are further described below in the Technical Disclosure. It may be understood that this Overview is not intended to identify key features or essential features of the claimed subject matter, nor is it intended to be used to limit the scope of the claimed subject matter.

### BRIEF DESCRIPTION OF THE DRAWINGS

Many aspects of the disclosure may be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an operational environment for providing a domain adaptation engine, according to an embodiment herein.

FIG. 2 illustrates an example domain adaptation engine, according to an embodiment provided herein.

FIG. 3 illustrates a process for providing a domain adaptation engine and its related functions, according to an embodiment herein.

FIG. 4 illustrates an example method for training the domain adaptation engine of FIG. 2, according to an embodiment herein.

FIG. 5 illustrates an example framework for a machine-learning system of the domain adaptation engine of FIG. 2, according to an embodiment herein.

FIG. 6 illustrates an example latent space representation, according to an embodiment herein.

FIG. 7 shows an example client device suitable for providing a domain adaptation engine and related functions, according to an embodiment herein.

### DETAILED DESCRIPTION

Early and accurate detection of diseased tissue is playing an increasingly central role in guiding treatment decisions and improving outcomes across a broad range of clinical applications. While traditionally focused on diagnosing conditions such as cancer, these detection methods are now also used to monitor transplant rejection and assess the severity of autoimmune diseases. Minimally invasive approaches—such as blood-based assays—are particularly valuable, as they reduce patient risk and support ongoing, real-time monitoring. The key challenge lies not only in detecting the presence of diseased tissue but also in accurately identifying its tissue-of-origin, cell-type-of-origin or treatment related disease signals to inform precise diagnosis and targeted therapy. It should be appreciated that, although the following discussion refers to “tissue-of-origin” for ease of explanation, the term is intended to broadly encompass not only tissue-of-origin, but also cell-type-of-origin and treatment-related disease signals.

One emerging strategy for addressing this challenge is the use of epigenomic profiling, particularly through techniques such as ATAC-Seq (Assay for Transposase-Accessible Chromatin using sequencing). Different cell types and tissues exhibit unique patterns of chromatin accessibility, which reflect the location of active regulatory elements and transcription factor binding sites. These patterns are tightly linked to gene expression and cellular identity. ATAC-Seq leverages this by mapping regions of open chromatin to infer the regulatory landscape of a sample. When applied to diseased tissue, such as a tumor, this method enables inference of the tissue-of-origin by comparing chromatin profiles to reference datasets. ATAC-Seq performed on sorted cells or Single-cell ATAC-Seq (scATAC-Seq) further enhances this capability by resolving cellular heterogeneity within complex samples, offering high-resolution insight into tissue-specific gene regulation. These methods are particularly valuable for tumors that are morphologically ambiguous or have undergone dedifferentiation, and they support more precise diagnosis and personalized treatment planning.

However, ATAC-Seq has numerous limitations that currently restrict its clinical use. Current ATAC-Seq methods require a high-quality biopsy of the diseased tissue, which may be invasive or infeasible depending on the tumor's location or patient health. Interpretation also depends on comparison with curated epigenomic reference maps and requires involvement from specialized computational teams and pathologists. As a result, ATAC-Seq and scATAC-Seq, while powerful in research settings, are not yet scalable for routine diagnostics.

To address the limitations of tissue biopsies, researchers have turned to cell-free DNA (cfDNA) as a minimally-invasive alternative. CfDNA consists of DNA fragments released into the bloodstream by dying cells, including both healthy and diseased tissues. Importantly, these fragments retain features that reflect the chromatin structure of their tissue-of-origin. As such, cfDNA provides an opportunity to assess tissue health and detect pathological changes through a simple blood draw. This has led to the development of cfDNA-based liquid biopsies for detecting cancer, monitoring organ transplant rejection, and assessing autoimmune disease activity. Because cfDNA sampling is minimally invasive, it enables repeat testing and broad applicability in clinical settings where traditional biopsy is impractical.

Despite these advantages, cfDNA-based detection methods face important technical and biological constraints. Most cfDNA in circulation originates from healthy cells, while the proportion derived from diseased tissue can be extremely low, often just 0.1% or less. Traditional cfDNA assays typically target known mutations within a small subset of genomic regions, discarding much of the sample. This limited scope reduces sensitivity and necessitates larger sample volumes—often up to 100 mL of blood—to achieve adequate detection. Moreover, cfDNA analysis is generally most effective in later-stage diseases where tumor burden is high and DNA shedding is more pronounced. Many early-stage cancers or diseased tissues with low turnover or poor vascular access release insufficient cfDNA to be reliably detected. Even more fundamentally, developing comprehensive tissue-of-origin models based on cfDNA requires extensive reference datasets representing every relevant tissue and disease state—an impractical and resource-intensive undertaking. These factors constrain the clinical utility of cfDNA as a broad diagnostic tool, particularly for early detection and diseases with less characterized genomic signatures.

To address the limitations in detecting and characterizing diseased tissue, an example domain adaptation engine is provided herein. The domain adaptation engine bridges the gap between high-resolution epigenomic techniques like ATAC-Seq and minimally invasive cfDNA-based liquid biopsies. While ATAC-Seq offers detailed tissue-of-origin information, it depends on invasive sampling and complex analysis. Conversely, cfDNA provides a minimally invasive alternative with broad clinical reach but suffers from sensitivity limitations and incomplete genomic coverage. The domain adaptation engine combines the strengths of both methods-leveraging the comprehensive epigenomic profiling capabilities of ATAC-Seq while maintaining the clinical accessibility of cfDNA-based liquid biopsies. This integrated approach enables more sensitive detection of diseased tissue at earlier stages, improves tissue-of-origin identification, and expands the range of conditions that can be effectively monitored through minimally invasive means.

In particular, the domain adaptation engine integrates chromatin-informed models with cfDNA fragmentation data to provide improved diagnostics that are both precise and clinically scalable. The domain adaptation engine leverages the detailed epigenetic profiling capabilities of ATAC-Seq for identifying tissue-of-origin using minimally invasive cfDNA samples. As will be expanded on in greater detail below, the domain adaptation engine includes a machine learning (ML) system containing one or more ML models that are trained to translate between ATAC-Seq and cfDNA data. Specifically, the domain adaptation engine deconvolutes the cfDNA and maps it to ATAC-Seq datasets to identify tissue-of-origin, enabling detection of diseased tissue at lower fractions, sometimes significantly lower, within a sample.

By translating between cell-type-specific ATAC-Seq and cfDNA data, the domain adaptation engine provides numerous advantages over conventional approaches to tumor detection and tissue-of-origin predictions. The domain adaptation engine leverages the abundant and diverse cell-type-specific ATAC-Seq datasets available from various tissue types to learn tissue-specific chromatin patterns. Rather than focusing on a small subset of genomic regions or specific mutations, this approach utilizes a larger spectrum of genomic information to detect diseased tissue. By training on the extensive data source provided by ATAC-Seq data, the domain adaptation engine captures the nuances of chromatin accessibility across different cell types and tissues, creating a comprehensive reference for tissue-specific patterns.

The domain adaptation engine may then be applied to detect diseased tissue and predict tissue-of-origin from cfDNA samples using fragmentomics approaches. When presented with a new cfDNA fragmentation pattern, the domain adaptation engine may analyze one or more physical and structural features of the DNA fragments—including fragment size or length distributions, end motifs, and breakpoint patterns—and detects similarities to the tissue-specific chromatin patterns learned from ATAC-Seq data. By matching these fragmentomic signatures (e.g., a diseased tissue signature) to specific tissue types, the domain adaptation engine infers the likely tissue-of-origin for the cfDNA fragments. This approach enables detection of historically difficult-to-detect tumor types, such as those that shed lower amounts of DNA or tumors with no mutations, because the domain adaptation engine detects regional activity and structural characteristics within the genome rather than identifying specific mutations. Additionally, the domain adaptation engine requires smaller sample sizes for detection purposes and allows for earlier disease detection when the diseased tissue represents only a small fraction of the total cfDNA.

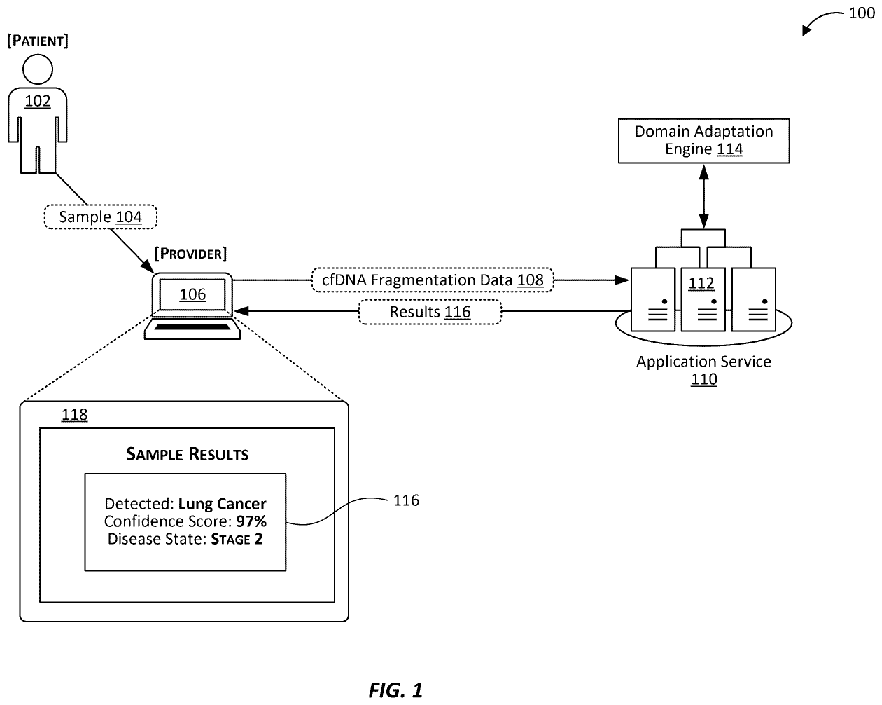

Turning now to FIG. 1, FIG. 1 illustrates an operational environment 100 for providing a domain adaptation engine, according to an embodiment herein. In particular, the environment 100 illustrates the domain adaptation engine 114 as leveraged by a provider to detect a diseased tissue, and predict a tissue-of-origin for the diseased tissue, or disease signature of a patient 102. For ease of discussion, the patient 102 presents in a clinical setting with a range of symptoms and is seeking medical evaluation from the provider.

As will be expanded on in greater detail below, the symptoms may be caused by an underlying disease that has not yet been definitively diagnosed. In the illustrated example, the patient 102 has lung cancer, however, it should be understood that the described systems and methods are equally applicable to a wide range of diseases, including but not limited to breast cancer, colorectal cancer, prostate cancer, liver cancer, kidney cancer, stomach cancer, autoimmune diseases, and transplant rejection. While the following description focuses on the use of a domain adaptation engine 114 as a diagnostic tool for detecting diseased tissue and determining tissue-of-origin, the domain adaptation engine 114 may also be applied to other clinical objectives. These include evaluating the likelihood of transplant rejection, monitoring disease progression, and assessing treatment response by analyzing changes in cell-free DNA (cfDNA) signatures over time.

Under conventional diagnostic workflows, confirming a disease such as lung cancer typically requires a tissue biopsy. However, obtaining a biopsy involves two key limitations: (A) the provider needs to first know where to biopsy (e.g., the lung) which often depends on prior imaging or clinical suspicion, and (B) the disease must be sufficiently advanced to be detectable through such means. These constraints can delay diagnosis, particularly in cases of early-stage or anatomically inaccessible diseases. Even advanced molecular techniques such as ATAC-seq, which can identify tissue-of-origin based on chromatin accessibility patterns, face practical limitations. ATAC-seq requires a high-quality tissue sample obtained through an invasive biopsy, which may be infeasible in patients with poor clinical status or tumors located in high-risk areas. Moreover, interpreting ATAC-seq data necessitates specialized computational infrastructure and expert analysis to compare results against comprehensive reference epigenomic datasets. As a result, despite its diagnostic potential, ATAC-seq remains largely confined to research settings and is not viable for widespread clinical deployment.

While the provider could alternatively utilize a cfDNA sample as part of the diagnostic workflow, conventional cfDNA-based approaches also present numerous limitations for reliably detecting lung cancer in the patient 102. Standard cfDNA assays typically rely on detecting known somatic mutations associated with specific cancers. Accordingly, accurate detection requires that the patient's lung cancer harbors a mutation that not only maps to a well-characterized genomic region but is also covered by the targeted panel used in the assay. Moreover, because cfDNA is derived from a mixture of both healthy and diseased cells, the proportion of cancer-derived cfDNA—often referred to as the tumor fraction—may be extremely low, especially in early-stage disease or tumors with limited vascular access. As a result, reliable mutation detection often depends on the cancer being sufficiently advanced so that enough tumor DNA is shed into the bloodstream. Without a sufficiently high tumor fraction, the signal may fall below the assay's sensitivity threshold, resulting in false negatives or inconclusive results. These constraints limit the effectiveness of conventional cfDNA assays in detecting disease early or in cases lacking canonical driver mutations.

Accordingly, the provider in the operational environment 100 leverages the domain adaptation engine 114 to enable disease detection and tissue-of-origin identification using a cfDNA sample 104. Rather than relying solely on mutation detection, the domain adaptation engine 114 analyzes fragmentation patterns present in the cfDNA sample 104, which retain structural features reflective of the chromatin accessibility landscape of the originating tissues. These fragmentation patterns are compared against reference chromatin accessibility signatures derived from ATAC-seq data, which capture tissue-specific regulatory architecture. In effect, the domain adaptation engine 114 performs a cross-domain translation, mapping the cfDNA fragmentation profile of the cfDNA sample 104 into the ATAC-seq domain. This transformation allows the domain adaptation engine 114 to predict the likely tissue source of the cfDNA fragments and identify potential diseased tissue types, thereby supporting diagnosis without the need for an invasive tissue biopsy.

To perform the domain translation described above, the cfDNA sample 104 may first be collected and processed to generate cfDNA fragmentation data 108. As illustrated, the patient 102 provides the sample 104—typically a blood draw—to a healthcare provider, which is represented by a client device 106 in the operational environment 100. While the diagram illustrates direct input to the client device 106, this is intended to represent the standard clinical workflow in which a blood sample is collected from the patient 102 in a laboratory or clinical setting. The collected blood sample is then processed to isolate plasma, from which cfDNA is extracted using protocols such as centrifugation and column-based purification. The resulting cfDNA is then subjected to high-throughput sequencing, such as whole-genome sequencing (WGS) or targeted sequencing, to capture millions of short DNA fragments.

The sequencing output is then processed to generate the cfDNA fragmentation data 108, which includes information about the size distribution of DNA fragments, the genomic coordinates of fragment start and end sites, and nucleotide patterns at fragment ends. These features collectively reflect the chromatin structure and nucleosome positioning of the cells from which the cfDNA originated. In some implementations, the cfDNA fragmentation data 108 may also include inferred fragment coverage profiles across the genome, relative enrichment of fragments around transcription start sites (TSS), and end-motif frequencies—all of which carry tissue-specific epigenetic signals.

Once generated, the provider, such as via the client device 106, may submit the cfDNA fragmentation data 108 to the domain adaptation engine 114 for processing. As illustrated, to submit the cfDNA fragmentation data 108 to the domain adaptation engine 114, the client device 106 may be in operable communication with an application service 110 for one or more functions or features. Broadly speaking, the application service 110 provides software application services to end points, such as the client device 106, examples of which include medical software for diagnosis purposes, generating treatment plans, or recording medical events for patients, such as the patient 102.

In the illustrated example, the application service 110 may provide a diagnostic tool for detecting diseased tissues for patients, such as for the patient 102. As such, the client device 106 may load and execute software applications locally that interface with services and resources provided by the application service 110, such as the domain adaptation engine 114. The applications may be natively installed and executed applications, web-based applications that execute in the context of a local browser application, mobile applications, streaming applications, or any other suitable type of application. Example services and resources provided by the application service 110 include front-end servers, application servers, content storage services, authorization and authentication services, and the like.

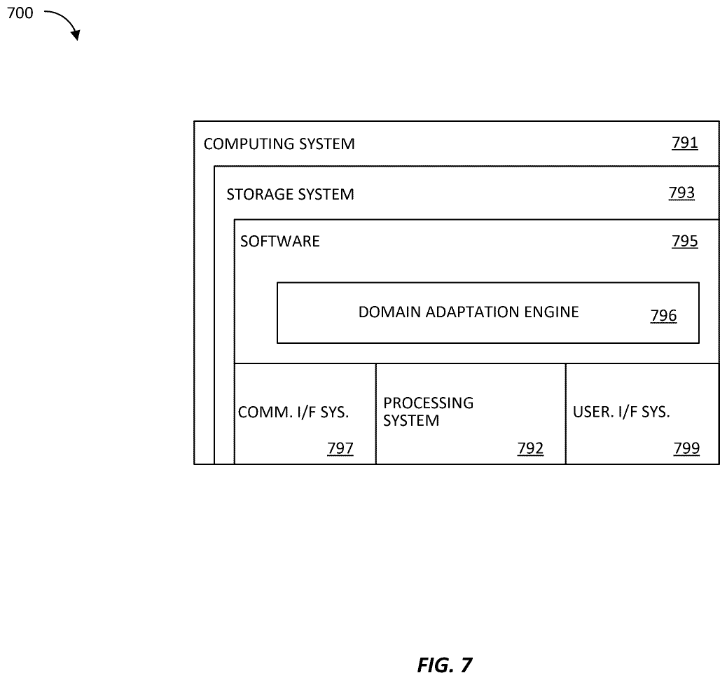

To interact with the application service 110, the client device 106 may communicate with the application service 110 via one or more internets and intranets, the Internet, wired and wireless networks, local area networks (LANs), wide area networks (WANs), or any other type of network or combination thereof. Examples of the client device 106 may include personal computers, tablet computers, mobile phones, gaming consoles, wearable devices, Internet of Things (IoT) devices, and any other suitable devices, of which computing apparatus 791 in FIG. 7 is also broadly representative.

In the illustrated example, the application service 110 operates in a cloud-based environment. As such, the application service 110 employs one or more server computers 112 co-located with respect to each other or distributed across one or more data centers to deliver its functionalities and services. Example servers include web servers, application servers, virtual or physical servers, or any combination or variation thereof, of which computing apparatus 791 in FIG. 7 is broadly representative.

As illustrated, the application service 110 may include an integration with the domain adaptation engine 114 for detecting diseased tissues and generating tissue-of-origin predictions, such as detecting cancer in the sample 104 from the patient 102 and identifying that the tissue-of-origin of the cancer is the lung. In some embodiments, the domain adaptation engine 114 may be executed remotely by the application service 110 or a third party, while in other embodiments the domain adaptation engine 114 may be installed and executed locally on the client device 106. In still other embodiments, one or more functions of the domain adaptation engine 114, as described herein, may be installed and executed locally on the client device 106, while the remaining functions are integrated and executed remotely via the application service 110 or a third party.

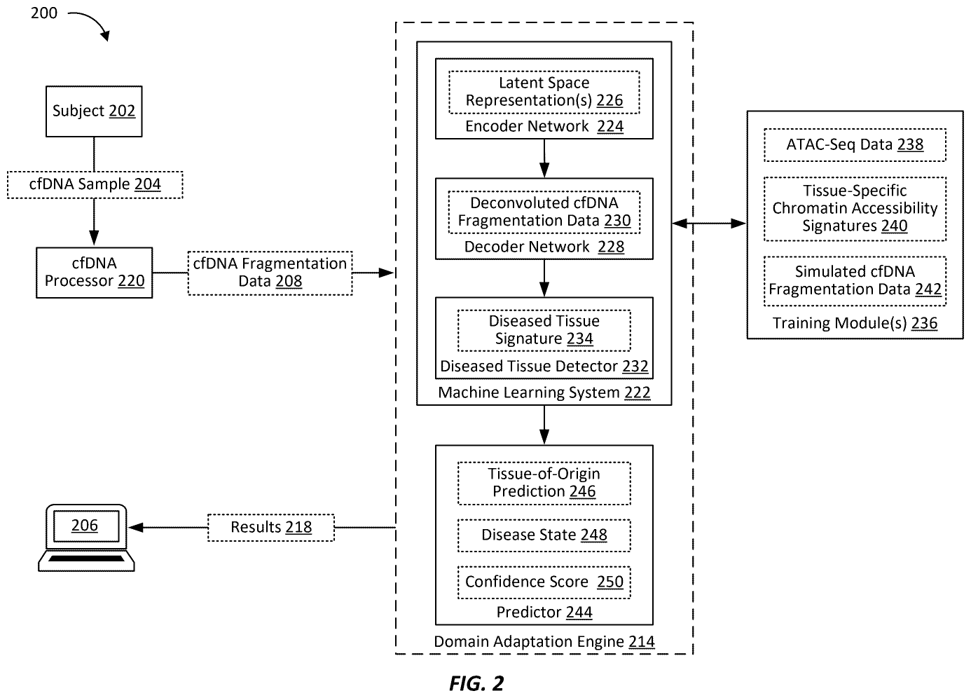

As noted above, for diagnostic purposes, the cfDNA fragmentation data 108 is submitted to the domain adaptation engine 114. Responsive to receiving the cfDNA fragmentation data 108, the domain adaptation engine 114 deconvolutes the cfDNA fragmentation data 108 to detect any diseased tissue signatures present in the sample 104, and based on a detected diseased tissue signature, predicting a tissue-of-origin. The deconvolution, detection, and prediction processes are described in greater detail below with respect to FIGS. 2-6.

In some embodiments, upon processing the cfDNA fragmentation data 108, the domain adaptation engine 114 may generate results 116 summarizing the outcome of its analysis. These results 116 may be transmitted to the client device 106 and displayed via a user interface 118 accessible to the provider. As illustrated, the results 116 may include an identification of the tissue-of-origin of the diseased tissue, such as lung cancer in the present example, a confidence score quantifying the certainty of the prediction, and a predicted disease state or classification (e.g., malignant vs. benign, or early- vs. late-stage disease). In certain implementations, the results 116 may also include visualization of cfDNA fragmentation patterns, comparisons to reference ATAC-seq signatures, or trend data reflecting changes over time in response to treatment. As will be described in greater detail below, the specific content and format of the results 116 may vary depending on the clinical application, diagnostic objective, or configuration of the domain adaptation engine 114.

By enabling disease detection and tissue-of-origin prediction using the cfDNA sample 104, the domain adaptation engine 114 allows the provider to accurately identify diseased tissue and develop an appropriate treatment plan for the patient 102, all without the need for invasive biopsies. This minimally-invasive approach is particularly beneficial in cases where the diseased tissue is anatomically inaccessible, the patient 102 is not a candidate for surgery, or conventional imaging fails to localize the disease. Moreover, the domain adaptation engine 114 enhances diagnostic sensitivity by analyzing fragmentation patterns, allowing for the detection of diseases in early stages or those that exhibit low cfDNA shedding rates, such as certain solid tumors or autoimmune conditions. By leveraging tissue-specific chromatin accessibility signatures, the domain adaptation engine 114 can identify subtle signals in cfDNA that would be missed by traditional mutation-based assays, enabling earlier intervention and more personalized care.

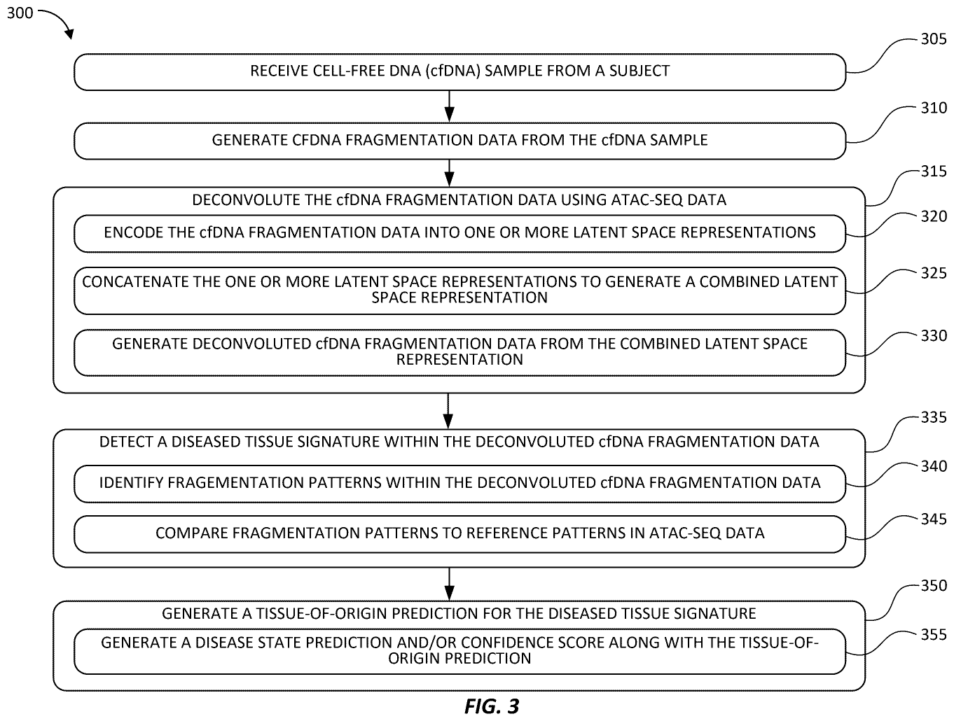

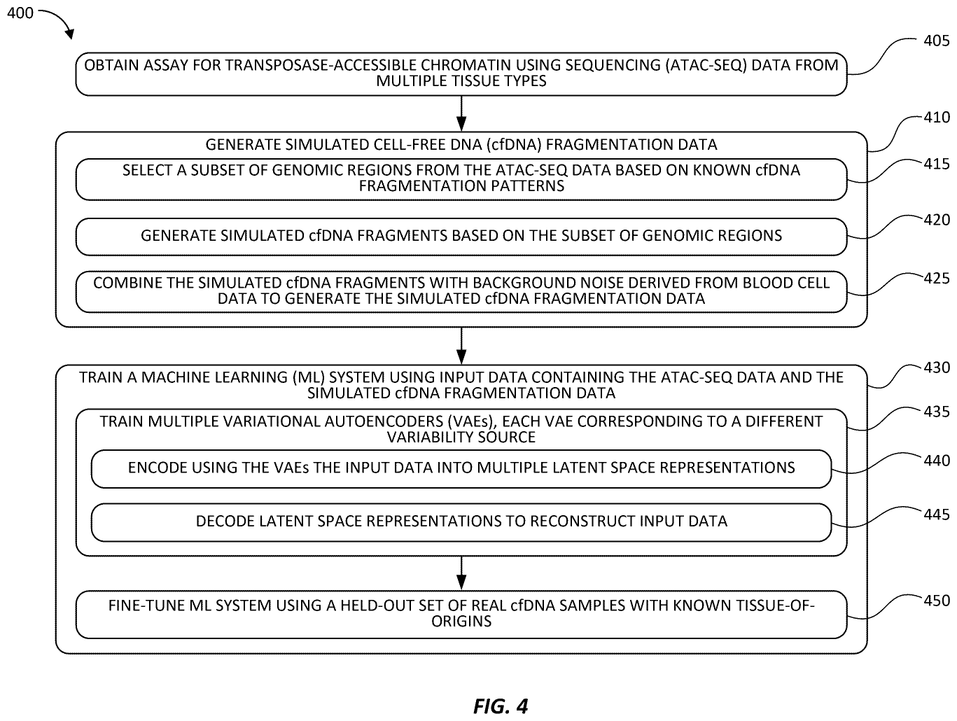

Referring now to FIG. 2, an example domain adaptation engine 214 is provided, according to an embodiment herein. For ease of illustration, FIG. 2 is described with respect to FIG. 3, which provides a process 300 for providing a domain adaptation engine and its related functions, such as the domain adaptation engine 214, according to an embodiment herein. Although FIG. 3 is described in relation to FIG. 2, it should be appreciated that the process 300 illustrated in FIG. 3 is equally applicable to other embodiments and components shown in the remaining figures. Additionally, FIGS. 2 and 3 are discussed in conjunction with FIGS. 4-6 for illustrative purposes. However, FIGS. 2 and 3 are not limited to the specific examples, components, or configurations depicted in FIGS. 2-6 and other variations are contemplated.

As illustrated, a subject 202 may submit a cfDNA sample 204—similar to the patient 102 and sample 104 described above—to a cfDNA processor 220 (305). For example, the cfDNA sample 204 may be a liquid biological sample, such as a blood sample, collected in a clinical setting. In some embodiments, the cfDNA sample 204 may have a volume ranging from approximately 2 mL to 25 mL, such as from 5 mL to 25 mL, from 2 mL to 10 mL, from 2 mL to 5 mL, or in preferred embodiments 5 mL. The specific volume may vary depending on the intended diagnostic sensitivity, the suspected disease type, or the expected abundance of cfDNA from diseased tissue.

Responsive to receiving the cfDNA sample 204, the cfDNA processor 220 may extract and sequence the cfDNA to generate cfDNA fragmentation data 208 (310). To generate the cfDNA fragmentation data 208, the cfDNA processor 220 may first isolate plasma from the cfDNA sample 204 through centrifugation, followed by extraction and purification of cfDNA using standard protocols such as silica column-based methods or magnetic bead separation. The purified cfDNA may then undergo high-throughput sequencing, such as WGS or shallow WGS, to capture the genomic coordinates, fragment lengths, and sequence characteristics of individual cfDNA molecules. This sequencing data may be further processed to compute fragmentation metrics, such as fragment length distribution, genomic start and end sites, end motifs, and coverage around regulatory elements like TSS, resulting in cfDNA fragmentation data 208. In some embodiments, the sequencing data may be processed to identify one or more of the fragmentation metrics or features, such as fragment length distribution.

It should be appreciated that while the cfDNA processor 220 is illustrated as functionally distinct from the domain adaptation engine 214, in some embodiments the cfDNA processor 220 may be integrated within the domain adaptation engine 214. Alternatively, both the cfDNA processor 220 and the domain adaptation engine 214 may be part of a common computational framework, such as the application service 110, enabling seamless end-to-end processing from raw biological input to tissue-of-origin prediction and diagnostic output.

Once generated, the cfDNA fragmentation data 208 may be provided to the domain adaptation engine 214, which may be the same or similar to the domain adaptation engine 114. Responsive to receiving the cfDNA fragmentation data 208, the domain adaptation engine 214 may deconvolute the cfDNA fragmentation data 208 using ATAC-Seq data, such as cell-type-specific ATAC-Seq data 238 (315). The cell-type-specific ATAC-Seq data 238 may correspond to multiple different tissue or cell types, such as lung, breast, liver, kidney, colon, prostate, pancreas, stomach, heart, brain, and hematopoietic tissues. Each tissue type exhibits a unique chromatin accessibility profile, reflecting differences in regulatory element activity and nucleosome positioning. As further detailed below, by deconvoluting the cfDNA fragmentation data 208, the domain adaptation engine 214 can identify distinct fragmentation patterns within the cfDNA sample 204 that correspond to known chromatin accessibility signatures 240 derived from the cell-type-specific ATAC-Seq data 238.

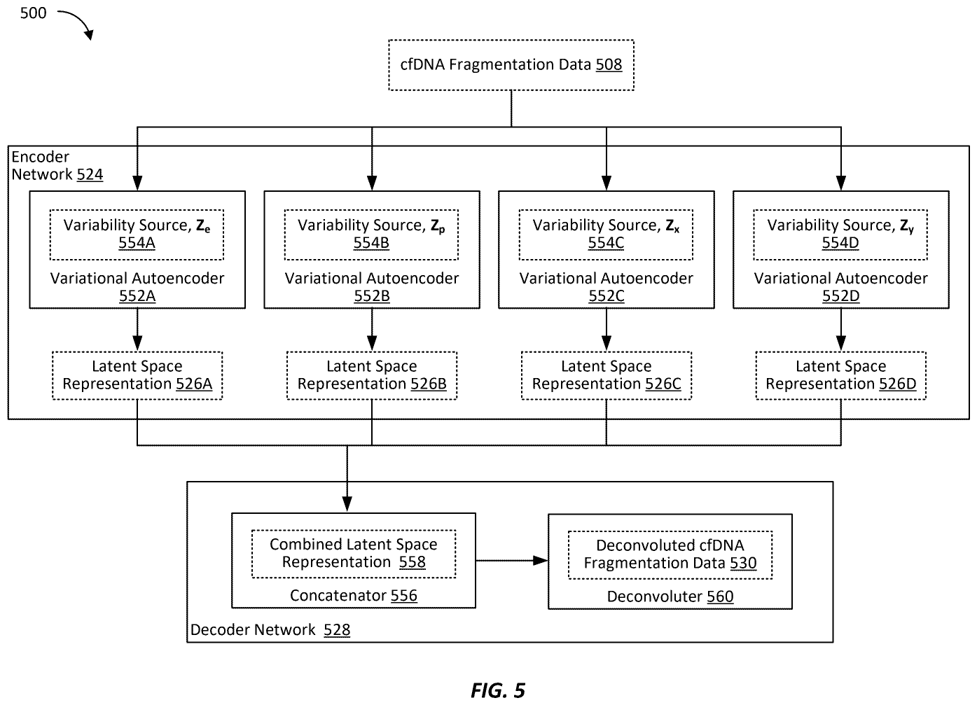

To deconvolute the cfDNA fragmentation data 208, the domain adaptation engine 214 may include a ML system 222. The ML system 222 may include a collection of ML models that operate in coordination to deconvolute the cfDNA fragmentation data 208. In some embodiments, the ML system 222 may include an encoder network 224 and a decoder network 228, each of which are described in greater detail below with respect to FIG. 5. To deconvolute the cfDNA fragmentation data 208, the ML system 222 may encode the cfDNA fragmentation data 208 into one or more latent space representations 226 (320). In particular, the encoder network 224 may encode the cfDNA fragmentation data 208 into multiple latent space representations 226.

The encoder network 224 may include multiple autoencoders, each of which generates a respective latent space representation 226 in accordance with its training. Each latent space representation 226 may correspond to a probabilistic latent space for a different variability source. As will be described in greater detail with respect to FIGS. 4-5, each autoencoder may be trained for a different variability source. That is, the encoder network 224 may encode the cfDNA fragmentation data 208 according to different variability sources. A variability source may be a domain or category of biological or experimental factors that contribute to differences in data patterns between tissue types, diseases, and/or disease stages. These variable sources can include technology-specific effects, tissue-specific chromatin structures, blood cell-type proportions, or other biological variables that influence the observed cfDNA fragmentation patterns. By separately encoding and analyzing these different sources of variability, as described below, the domain adaptation engine 214 can effectively isolate and interpret the relevant fragmentation patterns within the cfDNA fragmentation data 208. This approach allows the domain adaptation engine 214 to account for and potentially remove confounding factors, enhancing its ability to accurately identify diseased tissue signatures and predict tissue-of-origins.

After the cfDNA fragmentation data 208 is encoded into the latent space representations 226, the domain adaptation engine 214 may generate deconvoluted cfDNA fragmentation data 230 from the latent space representations 226. In particular, the decoder network 228 may generate the deconvoluted cfDNA fragmentation data 230 from the latent space representations 226. In some embodiments, to generate the deconvoluted cfDNA fragmentation data 230, the decoder network 228 may concatenate the latent space representations 226 to generate a combined latent space representation (325) and generate the deconvoluted cfDNA fragmentation data 230 from the combined latent space representation (330). These steps are described in greater detail below with respect to FIG. 5.

Responsive to generating the deconvoluted cfDNA fragmentation data 230, the domain adaptation engine 214 may detect a diseased tissue signature 234 (335). That is, the ML system 222 may include a diseased tissue detector 232 that detects the diseased tissue signature 234 present within the deconvoluted cfDNA fragmentation data 230. To detect the diseased tissue signature 234, the diseased tissue detector 232 may identify fragmentation patterns within the deconvoluted cfDNA fragmentation data 230 (340). The deconvoluted cfDNA fragmentation data 230 may include various fragmentation patterns, such as fragment size distribute patterns, end motif patterns, or breakpoint patterns, each of which may reflect tissue-specific chromatin architecture and regulatory activity.

In some embodiments, the diseased tissue detector 232 may compare the fragmentation patterns identified within the deconvoluted cfDNA fragmentation data 230 to known tissue-specific chromatin accessibility signatures 240 (345). The tissue-specific chromatin accessibility signatures 240 may be derived from the ATAC-Seq data 238 and reflect characteristic chromatin accessibility profiles of specific tissues or cell types. By aligning the fragmentation features, such as fragment size or length distribution patterns, end motif patterns, and breakpoint patterns, within the deconvoluted cfDNA fragmentation data 230 to the tissue-specific chromatin accessibility signatures 240, the diseased tissue detector 232 may infer whether a diseased tissue signature 234 is present and identify its tissue-of-origin. In other embodiments, this comparison may be performed inherently by the diseased tissue detector 232 through machine learning, wherein the diseased tissue detector 232 is trained on fragmentation data labeled with corresponding tissue-specific chromatin accessibility signatures 240 derived from the ATAC-Seq data 238. In such cases, the diseased tissue detector 232 may learn to associate observed fragmentation patterns with disease-relevant chromatin accessibility signatures 240, enabling prediction at inference time without requiring explicit pattern matching. Further details regarding the training and implementation of the diseased tissue detector 232 are described below with respect to FIGS. 4-5.

If and when the diseased tissue signature 234 is detected, the domain adaptation engine 214 may generate a tissue-of-origin prediction 246 for the diseased tissue signature 234 (350). In some embodiments, the domain adaptation engine 214 may include a predictor 244 configured to generate the tissue-of-origin prediction 246 by analyzing the fragmentation patterns associated with the diseased tissue signature 234. The tissue-of-origin prediction 246 may indicate the specific tissue type from which the cfDNA corresponding to the diseased tissue signature 234 was shed. For example, the predictor 244 may classify the diseased tissue as originating from the lung, liver, breast, or another organ, based on the correlation between the fragmentation patterns and reference tissue-specific chromatin accessibility signatures 240. Accurately predicting the tissue-of-origin for the diseased tissue signature 234 may be important for diagnosing the subject 202 and selecting an appropriate treatment plan tailored to the specific disease context. This targeted diagnostic insight may improve treatment efficacy and enable earlier therapeutic intervention.

In some embodiments, along with generating the tissue-of-origin prediction 246, the predictor 244 may also generate a disease state prediction 248 and/or a confidence score 250 (355). The disease state prediction 248 may indicate the stage, progression, or biological activity of the disease based on features extracted from the fragmentation patterns within the diseased tissue signature 234. For example, the disease state prediction 248 may classify the disease as early-stage, late-stage, high-proliferation, or quiescent, depending on fragmentation characteristics such as increased short fragment frequency, abnormal end motif enrichment, or disrupted nucleosome spacing. The confidence score 250 may quantify the statistical certainty or model confidence associated with the tissue-of-origin prediction 246 and/or the disease state prediction 248, enabling the provider to assess the reliability of the output. These additional outputs may enhance clinical decision-making by providing not only localization of the disease but also insight into its severity and biological behavior.

It should be appreciated that the specific outputs generated by the domain adaptation engine 214, such as the tissue-of-origin prediction 246, the disease state prediction 248, and the confidence score 250, may vary depending on the type of analysis or detection being performed. For example, in scenarios where the domain adaptation engine 214 is used not for disease detection but to assess organ transplant compatibility, the outputs may include different information relevant to transplant monitoring. In such cases, the predictor 244 may generate outputs such as a donor-organ match score, a predicted risk of graft rejection, or early indicators of immune-mediated injury based on cfDNA fragmentation patterns corresponding to the transplanted organ. Similarly, in autoimmune disease contexts, the outputs may reflect tissue-specific immune activity or predicted flare severity. Accordingly, the structure and content of the outputs from the domain adaptation engine 214 may be dynamically configured based on the clinical objective and application context.

Responsive to generating the tissue-of-origin prediction 246, and, in some cases, the disease state prediction 248 and the confidence score 250, the domain adaptation engine 214 may provide results 216 to a client device 206. The results 216, which may be the same as or similar to the results 116 described above, may include information derived from the cfDNA sample 207, such as whether any diseased tissue was detected. If diseased tissue is detected, the results 216 may further include relevant diagnostic details, such as the tissue-of-origin prediction 246, the disease state prediction 248, and the associated confidence score 250. The client device 206, which may be the same as or similar to the client device 106, may correspond to a provider, technician, or other medical personnel involved in the diagnosis or treatment of the subject 202. As such, responsive to receiving the results 216, the client device 206 may display the results via a user interface for review, interpretation, and potential integration into a broader clinical decision-making process. In some embodiments, the user interface may include visualizations, risk stratification indicators, or links to recommended next steps based on the analysis performed by the domain adaptation engine 214.

As noted above, the domain adaptation engine 214—specifically, the ML system 222—is trained using cell-type-specific ATAC-Seq data 238 and a limited number of cfDNA fragmentation data to encode the remaining cfDNA fragmentation data 208 into latent space representations 226. These latent representations 226 are used to identify diseased tissue signatures 234 and generate tissue-of-origin predictions 246. To support this training process, the domain adaptation engine 214 is in operable communication with one or more training modules 236. The training modules 236 are configured to supply the ML system 222 with annotated training data, enabling it to learn the relationships between cfDNA fragmentation features or patterns and chromatin accessibility profiles from known tissues and disease states.

Turning now to FIG. 4, an example method 400 for training the domain adaptation engine 214 to perform one or more functions is illustrated, according to various embodiments herein. For ease of illustration, the method 400 is described with respect to FIG. 2. To initiate the method 400, cell-type-specific ATAC-Seq data 238 may be obtained from multiple tissue types (405). That is, the ATAC-Seq data 238 representing a wide range of tissue types, disease conditions, and cellular states may be provided to the training module(s) 236. The ATAC-Seq data 238 serves as the reference dataset from which tissue-specific chromatin accessibility signatures 240 are derived. The tissue-specific chromatin accessibility signatures 240 capture regulatory features such as nucleosome positioning, open chromatin regions, and transcription factor binding sites that are unique to specific cell types and disease contexts. By learning from the ATAC-Seq data 238 during training, the ML system 222 is able to generalize to new cfDNA samples 204 and accurately detect abnormal fragmentation patterns indicative of diseased tissue and predict their tissue of origin.

In some embodiments, the training module(s) 236 may utilize the ATAC-Seq data 238 to generate simulated cfDNA fragmentation data 242 (410). The simulated cfDNA fragmentation data 242 can be used to train or pre-train the ML system 222 within the domain adaptation engine 214, especially when real cfDNA datasets with known ground-truth labels are limited or unavailable. The simulation process is designed to mimic the biological characteristics of cfDNA, including its fragmentation patterns and tissue-specific origin signals, enabling supervised or semi-supervised learning under controlled conditions.

To generate the simulated cfDNA fragmentation data 242, the training module(s) 236 may first select a subset of genomic regions from the ATAC-Seq data 238 based on known cfDNA fragmentation characteristics (415). These characteristics may include preferential cleavage around nucleosome-depleted regions, enrichment around TSS, and typical fragment lengths. The subset of genomic regions may correspond to open chromatin regions identified in a particular tissue type, thereby preserving the chromatin accessibility signal that is detectable in real cfDNA.

Using the selected regions, the training module(s) 236 may generate simulated cfDNA fragments by modeling the fragmentation process observed in vivo, which is primarily driven by apoptosis-related cleavage between nucleosomes (420). This may involve stochastically generating fragment start and end coordinates around accessible regions while enforcing empirically observed size distributions and nucleotide end motifs characteristic of cfDNA. These fragments may be computationally labeled with their tissue-of-origin based on the source tissue from the ATAC-Seq reference.

To further enhance biological realism, the training module(s) 236 may incorporate background noise derived from blood cell data—such as cfDNA fragmentation profiles from hematopoietic cells, which represent the dominant background signal in clinical plasma samples. By mixing the simulated tissue-specific fragments with this background signal, the training module(s) 236 can generate simulated cfDNA fragmentation data 242 that closely resembles real patient-derived cfDNA datasets, both in signal-to-noise ratio and in fragment-level characteristics.

By generating the simulated cfDNA fragmentation data 242, the training module(s) 236 enable the ML system 222 to learn robust and generalizable representations that distinguish tissue-specific signals from background noise. This is especially important for modeling real-world cfDNA samples, where disease-derived fragments are present at low fractional abundance and are often obscured by cfDNA originating from hematopoietic or other non-diseased cells. The simulated cfDNA fragmentation data 242 allows the ML system 222 to learn to recognize subtle fragmentation patterns that correlate with specific tissue-of-origin profiles and chromatin accessibility states.

This simulated data 242 is used to train components of the domain adaptation engine 214, including the diseased tissue detector 232, the predictor 244, and potentially other submodules. Training on these controlled, synthetic examples improves the domain adaptation engine's 214 ability to detect diseased tissue signatures 234 and accurately generate tissue-of-origin predictions 246 under challenging conditions. Such conditions include early-stage disease, where the tumor burden is low, or in cases where the diseased tissue sheds cfDNA at minimal levels, resulting in high noise and low signal scenarios. The ability to learn from simulated data also reduces the dependency on large, labeled cfDNA datasets, which are often difficult to obtain with known ground-truth annotations.

In some embodiments, the training cfDNA fragmentation data used to train the ML system 222 may include a combination of simulated cfDNA fragmentation data 242, real cfDNA fragmentation data derived from experimentally obtained samples, and synthetically generated or “unrealistic” cfDNA fragmentation data. While the simulated cfDNA fragmentation data 242 and real cfDNA fragmentation data capture biologically plausible fragmentation patterns, the inclusion of synthetically exaggerated or non-physiological examples, referred to here as “unrealistic” data, may be an intentional design choice that facilitates more effective training. These unrealistic examples allow the ML system 222 to more clearly learn and separate the distinct latent spaces associated with different variability sources, such as tissue-specific chromatin accessibility, blood cell-type proportions, and technical artifacts.

By training on both realistic (e.g., the simulated cfDNA fragmentation data 242 and real cfDNA fragmentation data) and unrealistic data, the ML system 222 may be better equipped to generalize and accurately encode cfDNA fragmentation patterns into their respective latent representations, even under complex or noisy conditions. If training were restricted solely to realistic data, the variability across latent dimensions may become entangled or underrepresented, making it more difficult for the ML system 222 to learn clean, disentangled latent spaces. This approach improves the overall robustness and flexibility of the domain adaptation engine 214, particularly in capturing subtle signals associated with diseased tissue signatures 234 and generating reliable tissue-of-origin predictions 246. In some embodiments, both the simulated cfDNA fragmentation data 242 and the ATAC-Seq data 238 are used to train the ML system 222 (430). As described in greater detail below with respect to FIG. 5, the ML system 222 may include multiple variational autoencoders (VAEs), each designed to capture and model a distinct source of variability within the input data. These variable sources may include biological variability (e.g., tissue-specific differences), technical noise (e.g., sequencing artifacts), or domain-specific transformations (e.g., differences between cfDNA and ATAC-seq signal modalities).

Each VAE within the ML system 222 may be trained on a specific variable source using the combined training data derived from both the simulated cfDNA fragmentation data 242 and the ATAC-Seq data 238 (435). The objective is to isolate and learn independent latent representations for each variable source, allowing the ML system 222 to perform robust domain adaptation and cross-modal inference. For example, one VAE may be trained to model tissue-specific chromatin accessibility structures, capturing variability across different tissue types, while another VAE may be trained to represent variability in blood cell-type proportions, accounting for the hematopoietic background signal commonly present in cfDNA samples.

To train a given VAE, the simulated cfDNA fragmentation data 242 and the ATAC-Seq data 238 are provided as input to the encoder network 224 of the VAE, which encodes the input into a respective latent space representation (440). Each VAE learns a probabilistic latent space that captures variability along its designated axis, which may be the predefined variable source (e.g., tissue-of-origin, tumor burden, or sequencing technology). The same input data may be submitted to multiple VAEs in parallel, resulting in multiple latent representations—each within its respective latent space—corresponding to different modeled variable sources.

These latent representations are then passed through respective decoder networks, such as the decoder network 228, to reconstruct the original input data (445). During training, the parameters of the encoder and decoder networks 224/228 within each VAE, along with other hyperparameters of the ML system 222, are optimized to minimize reconstruction loss. Specifically, training continues until the reconstructed input data is substantially similar to the original input data or within a defined similarity threshold, as well as minimizing latent space specific errors, ensuring that the latent space accurately captures the structure of the input distribution. For example, training may continue until the total loss of the ML system 222 is minimized to an acceptable level, where the total loss is the sum of the loss of each VAE to predict a source of variability and the reconstruction loss of the ML system 222. This approach enables the ML system 222 to generalize to unseen cfDNA samples and accurately infer disease-relevant features, even when presented with noisy, incomplete, or cross-domain inputs.

In some embodiments, the training of each of the VAEs within the ML system 222 may involve different levels of supervision, depending on the nature and availability of labeled training data for each variable source. For example, at least one of the VAEs may undergo fully supervised training, in which both the input data and corresponding labels for the variable of interest are available. Another VAE may undergo semi-supervised training, where only a subset of the input data is labeled, and the model learns from both labeled and unlabeled examples. Additionally, at least one VAE may undergo unsupervised training, where no explicit labels are provided and the model infers latent structure directly from the input data.

In an illustrative implementation, the VAE trained to encode technology-specific variability, such as differences between cfDNA fragmentation and ATAC-Seq chromatin accessibility profiles, may undergo fully supervised training using paired cfDNA and ATAC-Seq data 238. Similarly, the VAE trained to model tissue-specific chromatin structure may also be fully supervised using labeled ATAC-Seq data 238 from known tissue types. In contrast, the VAE trained to model blood cell-type proportion variability may be trained in a semi-supervised manner, using partial labeling from hematopoietic reference datasets combined with unlabeled cfDNA data. A fourth VAE may be included to capture residual or unmodeled variability (e.g., inter-individual differences, epigenetic noise, or sequencing artifacts) and may be trained in a fully unsupervised fashion. This flexible, multi-level supervision framework allows the ML system 222 to disentangle complex biological and technical sources of variability and improve the robustness of downstream tasks such as disease detection and tissue-of-origin prediction.

Once the encoder network 228, including the VAEs, has been trained, the ML system 222 may be fine-tuned using a held-out set of real cfDNA samples with known tissue-of-origin labels (450). This fine-tuning process refines the latent representations and prediction layers to optimize performance on real-world biological data. Specifically, the ML system 222, in conjunction with the predictor 244, may be adjusted to generate tissue-of-origin predictions 246 that meet or exceed a predefined level of accuracy, such as a threshold classification accuracy, precision, recall, or confidence score, based on the held-out validation set. This fine-tuning phase ensures that the domain adaptation engine 214 generalizes well beyond the simulated training data and is capable of accurately interpreting real cfDNA fragmentation patterns in clinical samples, such as the cfDNA sample 204. The fine-tuning step serves to calibrate the latent embeddings learned from simulated cfDNA fragmentation data 242 and the ATAC-Seq data 238 against experimentally validated cfDNA samples, bridging the domain gap between synthetic and empirical data.

It should be appreciated that, while the predictor 244 is illustrated as a separate component from the ML system 222, in some embodiments the predictor 244 may be integrated as part of the ML system 222. For example, the predictor 244 may be implemented as a fully connected classification layer appended to the latent space of one or more VAEs, or as a shared output layer across multiple latent embeddings. This modular design allows the ML system 222 to remain flexible and extensible across various prediction tasks, such as disease state prediction 248 or transplant rejection risk, in addition to tissue-of-origin prediction 246.

Referring now to FIG. 5, an example framework 500 for the ML system 222 is illustrated, according to various embodiments herein. As shown, the framework 500 includes an encoder network 524 and a decoder network 528, which may be the same as or similar to the encoder network 224 and the decoder network 228, respectively. The encoder network 524 includes multiple variational autoencoders (VAEs) 552A-D, each configured to encode distinct sources of variability present in the input data, such as tissue-specific chromatin accessibility, blood cell-type proportions, technology-specific noise, or other residual variation.

A variational autoencoder (VAE) is a type of generative model that uses a probabilistic framework to learn compressed representations of input data in a latent space. Unlike standard autoencoders, which map input data to a fixed latent code, a VAE encodes the input into a probability distribution (typically a multivariate Gaussian). During training, the VAEs 552A-D optimizes both a reconstruction loss and a Kullback-Leibler (KL) divergence term to ensure that the learned latent distribution approximates a known prior (e.g., standard normal). This design allows the VAEs 552A-D to capture uncertainty in the input and enables smooth interpolation and sampling in the latent space, making them particularly well-suited for modeling biological variability and noisy high-dimensional data such as cfDNA fragmentation and chromatin accessibility profiles.

In other embodiments, alternative latent variable models or neural architectures may be used in place of VAEs. For example, standard autoencoders, denoising autoencoders, or normalizing flows may be employed depending on the desired balance of model complexity, interpretability, and generative capacity. Additionally, transformer-based encoders, contrastive learning models, or graph-based neural networks could be used where appropriate for capturing long-range dependencies, integrating multi-omic data, or modeling cell-type relationships. The choice of model architecture may vary based on application requirements, available training data, or specific sources of biological and technical variability to be modeled.

As noted above, the VAEs 552A-D are variational autoencoders, each trained to encode input data into a respective probabilistic latent space defined by a designated source of variability. Unlike deterministic encoders, each VAE 552A-D models the input as a probability distribution in latent space, typically by learning parameters of a multivariate Gaussian (i.e., mean and variance) that reflect uncertainty in the resulting representation. During training, the VAEs 552A-D are optimized to minimize both a reconstruction loss—ensuring the decoder network 528 can accurately reconstruct the input—and a regularization term (e.g., Kullback-Leibler divergence), which encourages the learned latent distribution to approximate a predefined prior, such as a standard normal distribution.

Each of the VAEs 552A-D is assigned a different variability source and trained to capture a specific dimension of variation in the input data. For example, the first VAE 552A may encode input data according to a first variability source, Zₑ 554A, corresponding to technology-specific variability, such as differences introduced by sequencing platforms, sample preparation protocols, or assay modalities (e.g., cfDNA versus ATAC-Seq). The second VAE 552B may encode input data according to a second variability source, Zₚ 554B, which captures tissue-specific variability, such as chromatin accessibility patterns unique to different tissue types. The third VAE 552C may encode the input data according to a third variability source, Zₓ 554C, which is dedicated to capturing residual or unexplained variability, including subject-specific differences, stochastic biological noise, or other latent factors not directly attributable to the primary sources of interest. Finally, the fourth VAE 552D may encode the input data according to a fourth variability source, Z_{γ} 554D, representing blood cell-type proportion variability, which accounts for the hematopoietic background signal commonly present in cfDNA samples due to contributions from white blood cells and other circulating immune cells.

As shown, when cfDNA fragmentation data 508—which may be the same as or similar to the cfDNA fragmentation data 208—is input into the encoder network 524, each of the VAEs 552A-D may encode the data into a respective latent space representation 526A-D. These latent space representations 526A-D correspond to the different variability sources learned by the VAEs, such that each representation captures a distinct aspect of the underlying structure in the cfDNA fragmentation data 508. The latent space representations 526A-D depict the cfDNA fragmentation data 508 within independent probabilistic latent spaces associated with their respective variability sources: technology-specific variability (Zₑ 554A), tissue-specific chromatin accessibility patterns (Zₚ 554B), residual or unexplained variability (Zₓ 554C), and blood cell-type proportion variability (Z_{γ} 554D). These latent representations 526A-D preserve the uncertainty and multi-dimensional structure of the input data while isolating specific signal components relevant to downstream tasks.

By decomposing the cfDNA fragmentation data 508 in this manner, the ML system 222 can more accurately perform domain adaptation, noise reduction, and feature disentanglement. This enables improved performance in detecting diseased tissue signatures 234, generating tissue-of-origin predictions 246, and supporting additional inference tasks such as disease state prediction 248 or transplant compatibility assessment.

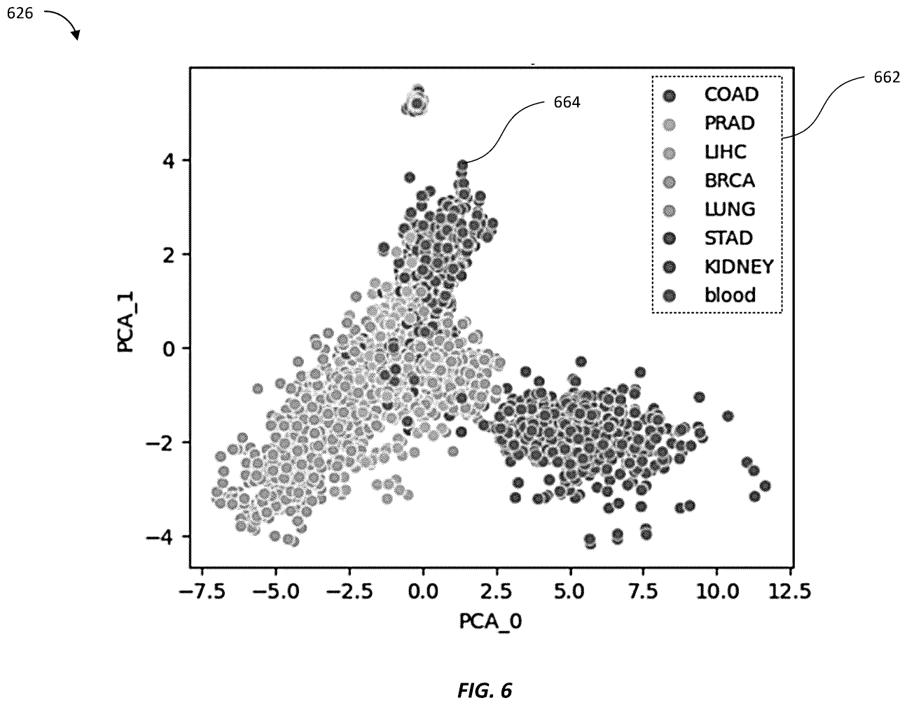

Referring briefly to FIG. 6, an example latent space representation 626 is illustrated, according to various embodiments herein. The latent space representation 626 provides a conceptual illustration of various tissue types 662 encoded into a probabilistic latent space by the second VAE 552B, which is trained to model tissue-specific chromatin accessibility variability (Z 554B). The data points 664 correspond to encoded representations of various cfDNA fragmentation data points, each projected into the latent space based on fragmentation features indicative of their tissue-of-origin.

In the illustrated example, distinct clusters of data points 664 may form within the latent space representation 626, with each cluster corresponding to a specific tissue type 662 based on chromatin accessibility patterns learned from the ATAC-Seq data 238. These patterns are encoded by the second VAE 552B, which captures tissue-specific variability (Z 554B). In this context, the tissue types 662 may include: COAD (colon tissue, representing colorectal adenocarcinoma), PRAD (prostate tissue, representing prostate adenocarcinoma), LIHC (liver tissue, representing hepatocellular carcinoma), LUNG (lung tissue, including lung adenocarcinoma and squamous cell carcinoma), BRCA (breast tissue, representing breast invasive carcinoma), STAD (stomach tissue, representing stomach adenocarcinoma), KIDNEY (kidney tissue, including renal clear cell, papillary, and chromophobe carcinomas), and Blood (hematopoietic tissue, including immune cells such as lymphocytes and leukocytes that contribute to the background cfDNA signal).

Each data point 664 in the latent space representation 626 reflects the encoded fragmentation signature of a cfDNA sample. The spatial proximity of these data points 664 indicates similarity in fragmentation characteristics and, by extension, their tissue-of-origin. Clusters representing different tissue types remain well-separated due to the distinct chromatin accessibility profiles captured by the ATAC-Seq data 238 and modeled by the VAE 552B. Overlapping or diffuse clusters may indicate either biological similarity or a mixed-origin cfDNA sample.

The probabilistic nature of the latent space enables the modeling of uncertainty in each data point's 664 representation, which is particularly important in clinical scenarios where the cfDNA sample may contain contributions from multiple tissues or where the diseased tissue is shedding DNA at very low levels. This capability allows the ML system 222 to produce soft predictions or distributional outputs that reflect varying degrees of confidence, rather than hard classifications.

The tissue-resolved latent space representation 626 enables the ML system 222, in conjunction with the predictor 244, to generate accurate tissue-of-origin predictions 246 by associating an input cfDNA fragmentation profile with the most probable tissue cluster. In some embodiments, the predictor 244 may also generate a confidence score 250 based on the density, separation, or entropy of the latent space distribution surrounding a given data point. This confidence score 250 provides a quantitative measure of prediction certainty, supporting clinical decision-making by allowing providers to weigh the reliability of the prediction when forming diagnostic or treatment plans.

Returning now to FIG. 5, once the latent space representations 526A-D are generated by each of the VAEs 552A-D, the decoder network 528 may reconstruct a deconvoluted version of the cfDNA fragmentation data 530. To accomplish this, the decoder network 528 may include a concatenator 556, which generates a combined latent space representation 558 by aggregating the individual latent space representations 526A-D. Each of these latent space representations 526A-D corresponds to a different learned variability source, such as tissue-specific chromatin accessibility, blood cell-type proportions, sequencing technology artifacts, or residual stochastic variation. By generating the combined latent space representation 558, the decoder network 528 integrates these independent representations 526A-D into a unified probabilistic encoding that captures the multi-source variability inherent in the input cfDNA fragmentation data 508.

The combined latent space representation 558 is then provided to a deconvoluter 560, which processes the integrated latent information to generate the deconvoluted cfDNA fragmentation data 530. This output represents a transformed version of the original cfDNA fragmentation data 508 in which confounding sources of variation have been computationally disentangled from tissue-specific disease signals. As a result, the deconvoluted cfDNA fragmentation data 530 enables clearer separation between cfDNA derived from healthy tissue and cfDNA originating from diseased tissue. This refinement improves the resolution and interpretability of downstream predictions. In particular, it enhances the accuracy of detecting the diseased tissue signature 234, as well as the generation of tissue-of-origin predictions 246 and disease state predictions 248. This deconvolution step may be important in clinical scenarios where disease-associated cfDNA is present at very low abundance and would otherwise be obscured by dominant background signals.

Building on this deconvolution capability, the domain adaptation engine 214 offers a robust and scalable framework for cfDNA-based disease detection and characterization. By leveraging a modular ML system 222 composed of multiple the VAEs 552A-D, the domain adaptation engine 214 systematically isolates and models diverse sources of biological and technical variability, including tissue-specific chromatin accessibility, blood cell-type composition, sequencing modality differences, and stochastic noise. Through latent space encoding, probabilistic inference, and cross-domain alignment with reference ATAC-Seq data 238, the domain adaptation engine 214 translates cfDNA fragmentation patterns into high-resolution, tissue-informed representations. This enables accurate identification of diseased tissue signatures 234 and tissue-of-origin predictions 246, even in early-stage disease or in cases where traditional mutation-based cfDNA assays lack sufficient sensitivity due to low tumor fraction or uncharacterized mutation profiles.

The ability of the domain adaptation engine 214 to operate on minimally invasive, blood-derived cfDNA samples, while accounting for biological complexity and technical noise, supports repeatable, high-sensitivity monitoring across a broad range of clinical use cases. These include cancer detection and localization, transplant rejection surveillance, autoimmune disease assessment, and treatment response monitoring. Furthermore, the domain adaptation engine's 214 modular architecture allows it to be adapted to new data types, disease indications, or analytical objectives without requiring full re-training of the entire ML system 222. Overall, the domain adaptation engine 214 represents an important advancement in cfDNA analysis by bridging the gap between high-dimensional epigenomic data and practical, clinically actionable diagnostics.

Referring to FIG. 7, FIG. 7 illustrates a computing apparatus 791 that may be used for providing a domain adaptation engine and related functions, as described herein. For example, the client devices 106 or 206, or the domain adaptation engine 114 or 214 may be or include the computing apparatus 791. As illustrated, the computing apparatus 791 includes a processing system 792 that includes a microprocessor and other circuitry that retrieves and executes software 795 from storage system 793. The processing system 792 may be implemented within a single processing device but may also be distributed across multiple processing devices or sub-systems that cooperate in executing program instructions. Examples of the processing system 792 include general purpose central processing units, graphical processing units, application specific processors, and logic devices, as well as any other type of processing device, combinations, or variations thereof.

The storage system 793 may comprise any computer-readable storage media or medium readable by processing system 792 and capable of storing software 795. The storage system 793 may include volatile and nonvolatile, removable and non-removable media implemented in any method or technology for storage of information, such as computer readable instructions, data structures, program modules, or other data. Examples of storage media include random access memory, read only memory, magnetic disks, optical disks, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations the storage system 793 may also include computer readable communication media over which at least some of the software 795 may be communicated internally or externally. The storage system 793 may be implemented as a single storage device but may also be implemented across multiple storage devices or sub-systems co-located or distributed relative to each other. The storage system 793 may comprise additional elements, such as a controller capable of communicating with the processing system 792 or possibly other systems.

The software 795 (including domain adaptation engine process 796) may be implemented in program instructions and among other functions may, when executed by the processing system 792, direct the processing system 792 to operate as described with respect to the various operational scenarios, sequences, and processes illustrated herein. For example, the software 795 may include program instructions for implementing a domain adaptation engine and related functions, such as the processes 300 and 400, as described herein.

The term “engine” as used herein includes a “component”, “module”, “system,” and the like is intended to refer to a computer-related entity, either software-executing general-purpose processor, hardware, firmware and a combination thereof. For example, an engine may be, but is not limited to being, a process running on a hardware processor, a hardware-based processor, an object, an executable, a thread of execution, a program, and/or a computer.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. The software 795 may include additional processes, programs, or components, such as operating system software, virtualization software, or other application software. The software 795 may also comprise firmware or some other form of machine-readable processing instructions executable by the processing system 792.

In general, the software 795 may, when loaded into the processing system 792 and executed, transforms a suitable apparatus, system, or device (of which computing apparatus 791 is representative) overall from a general-purpose computing system into a special-purpose computing system customized to generate features, functionality, and user experiences provided by the domain adaptation engine. Indeed, encoding the software 795 on the storage system 793 may transform the physical structure of the storage system 793. The specific transformation of the physical structure may depend on various factors in different implementations of this description. Examples of such factors may include, but are not limited to, the technology used to implement the storage media of the storage system 793 and whether the computer-storage media are characterized as primary or secondary storage, as well as other factors.

For example, if the computer readable storage media are implemented as semiconductor-based memory, the software 795 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 797 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

User interface system 799 may include various components and devices that enable interaction between the user and the computing system. Examples of these components and devices may include display screens, touchscreens, keyboards, mice, trackpads, styluses, voice recognition microphones, and other input/output devices. The user interface system 799 facilitates user commands and feedback through graphical user interfaces (GUIs), command-line interfaces (CLIs), or other interaction models. These interfaces may display information, receive user inputs, and provide visual, auditory, or tactile responses. The components and devices within the user interface system 799 are designed to ensure seamless and intuitive user interaction, leveraging well-established technologies and practices that need not be elaborated upon here.

Communication between the computing apparatus 791 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of network, or variation thereof. The aforementioned communication networks and protocols are well known and need not be discussed at length here.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example one or more non-transitory computer-readable media, which may store processor-executable instructions that, when executed by the processor, can cause the processor to perform methods according to this disclosure as carried out, or assisted, by a processor. Examples of may include, but are not limited to, an electronic, optical, magnetic, or other storage device capable of providing a processor, such as the processor in a web server, with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

Examples are described herein in the context of systems and methods for providing a domain adaptation engine and related functions. Those of ordinary skill in the art will realize that the foregoing description is illustrative only and is not intended to be in any way limiting. Reference is made in detail to implementations of examples as illustrated in the accompanying drawings. The same reference indicators will be used throughout the drawings and the following description to refer to the same or like items.

Additionally, the foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure. In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

### EXAMPLES

These illustrative examples are mentioned not to limit or define the scope of this disclosure, but rather to provide examples to aid understanding thereof. Illustrative examples are discussed above in the Detailed Description, which provides further description. Advantages offered by various examples may be further understood by examining this specification.

As used below, any reference to a series of examples is to be understood as a reference to each of those examples disjunctively (e.g., “Examples 1-4” is to be understood as “Examples 1, 2, 3, or 4”).

Example 1 is a computing apparatus comprising: a computer-readable storage media comprising processor-executable instructions stored thereon; and a processor coupled to the computer-readable storage media and configured to execute the processor-executable instructions that, when executed by the processor, direct the computing apparatus to at least: receive a cell-free DNA (cfDNA) sample from a subject; process the cfDNA sample to generate cfDNA fragmentation data; input the cfDNA fragmentation data into a pre-trained machine learning (ML) system, wherein the pre-trained ML system: (a) has been trained on cell-type-specific Assay for Transposase-Accessible Chromatin using sequencing (ATAC-Seq) data from multiple tissue or cell types, and (b) is configured to translate between ATAC-Seq data and cfDNA fragmentation data; analyze the cfDNA fragmentation data using the pre-trained ML system to identify tissue-specific chromatin accessibility patterns; predict a tissue-of-origin for the cfDNA sample based on the identified tissue-specific chromatin accessibility patterns; and generate a prediction of the tissue-of-origin for the cfDNA sample.

Example 2 is the computing apparatus of any previous or subsequent Example, wherein: the pre-trained ML system comprises a plurality of variational autoencoders (VAEs), each trained within a respective variability source of a plurality of variability sources; and the processor-executable instructions to analyze the cfDNA fragmentation data using the pre-trained ML system to identify tissue-specific accessibility patterns, when executed by the processor, further direct the computing apparatus to: encode, by the plurality of VAEs, the cfDNA fragmentation data into a plurality of latent spaces representations, wherein each VAE encodes the cfDNA fragmentation data into a respective latent space representations corresponding to a respective variability source; and generate deconvoluted cfDNA fragmentation data based on the cfDNA fragmentation data as encoded into the plurality of latent space representations.

Example 3 is the computing apparatus of any previous or subsequent Example, wherein the plurality of variability sources that the plurality of VAEs are trained on comprises one or more of: a technology-specific variability source; a tissue type variability source; a blood cell-type proportion variability source; and a remaining variability source.

Example 4 is the computing apparatus of any previous or subsequent Example, wherein the processor-executable instructions to predict the tissue-of-origin for the cfDNA sample based on the identified tissue-specific chromatin accessibility patterns, when executed by the processor, further direct the computing apparatus to: analyze a plurality of fragmentation patterns within the cfDNA fragmentation data as processed by the pre-trained ML system, wherein the plurality of fragmentation patterns comprises one or more of fragment size distribution patterns, end motif patterns, or breakpoint patterns; compare the plurality of fragmentation patterns to reference patterns derived from ATAC-Seq data associated with known tissue types to identify tissue-specific chromatin accessibility signatures; and determine the tissue-of-origin prediction based on the identified tissue-specific chromatin accessibility signatures.

Example 5 is the computing apparatus of any previous or subsequent Example, wherein the pre-trained ML system comprises a neural network trained on paired cell-type-specific ATAC-Seq and cfDNA fragmentation data from known tissue types, wherein the neural network comprises: an encoder network configured to: receive training cfDNA fragmentation data as input; and encode the training cfDNA fragmentation data into a latent space representation; a decoder network configured to: receive the latent space representation from the encoder network; and reconstruct the ATAC-Seq data from the latent space representation; wherein the neural network is trained to minimize the difference between: the ATAC-Seq data as reconstructed in an output from the decoder network; and the paired ATAC-Seq data corresponding to the training cfDNA fragmentation data submitted as an input; and wherein the encoder network, as trained, is used to process the cfDNA fragmentation data to identify the tissue-specific chromatin accessibility patterns present within the cfDNA sample.

Example 6 is the computing apparatus of any previous or subsequent Example, wherein: the pre-trained ML system comprises a plurality of variational autoencoders (VAEs), each VAE corresponding to a different variability source, wherein each VAE is configured to: receive the cfDNA fragmentation data as input; and encode the cfDNA fragmentation data into a respective latent space representation; and the processor-executable instructions to analyze the cfDNA fragmentation data using the pre-trained ML system to identify the tissue-specific chromatin accessibility patterns, when executed by the processor, further direct the computing apparatus to: concatenate the latent space representations from each VAE of the plurality of VAEs to form a combined latent space representation; and process the combined latent space representation to identify the tissue-specific chromatin accessibility patterns.

Example 7 is the computing apparatus of any previous or subsequent Example, wherein the pre-trained ML system is configured to identify tissue-of-origin for at least three of: breast cancer; colorectal cancer; lung cancer; prostate cancer; liver cancer; kidney cancer; stomach cancer; autoimmune diseases; and organ transplant rejection.

Example 8 is a computer-implemented method for predicting tissue-of-origin from cell-free DNA (cfDNA) samples, wherein the method comprises: receiving, by a domain adaptation engine, a cell-free DNA (cfDNA) sample from a subject; generating, by the domain adaptation engine, cfDNA fragmentation data from the cfDNA sample; deconvoluting, by the domain adaptation engine, the cfDNA fragmentation data using Assay for Transposase-Accessible Chromatin using sequencing (ATAC-Seq) data from multiple tissue types to generate deconvoluted cfDNA fragmentation data; detecting, by the domain adaptation engine, a diseased tissue signature within the deconvoluted cfDNA fragmentation data; and generating, by the domain adaptation engine, a tissue-of-origin prediction for the diseased tissue signature based on the deconvoluted cfDNA fragmentation data.

Example 9 is the method of any previous or subsequent Example, wherein deconvoluting, by the domain adaptation engine, the cfDNA fragmentation data using ATAC-Seq data comprises: encoding, by the domain adaptation engine, the cfDNA fragmentation data into a plurality of latent space representations, wherein each latent space representation corresponds to a respective variability source of a plurality of variability sources; and generating, by the domain adaptation engine, the deconvoluted cfDNA fragmentation data based on the cfDNA fragmentation data as encoded into the plurality of latent space representations.

Example 10 is the method of any previous or subsequent Example, wherein the plurality of variability sources comprises one or more of: a technology specific variability source; a tissue type variability source; a blood cell-type proportion variability source; and a remaining variability source.

Example 11 is the method of any previous or subsequent Example, wherein the method further comprises: generating, by the domain adaptation engine, a confidence score associated with the tissue-of-origin prediction; and transmitting, by the domain adaptation engine, the confidence score along with the tissue-of-origin prediction to a client device, wherein the confidence score and the tissue-of-origin prediction are displayed via a user interface of the client device.

Example 12 is the method of any previous or subsequent Example, wherein detecting, by the domain adaptation engine, the diseased tissue signature within the deconvoluted cfDNA fragmentation data comprises: analyzing, by the domain adaptation engine, a plurality of fragmentation patterns within the deconvoluted cfDNA fragmentation data, wherein the fragmentation patterns comprise one or more of fragment size distribution patterns, end motif patterns, or breakpoint patterns; comparing, by the domain adaptation engine, the plurality of fragmentation patterns to reference patterns derived from ATAC-Seq data associated with known tissue types to identify tissue-specific chromatin accessibility signatures; and identifying, by the domain adaptation engine, the diseased tissue signature based on deviations in the identified tissue-specific chromatin accessibility signatures from expected patterns of healthy tissue.

Example 13 is the method of any previous or subsequent Example, wherein: the domain adaptation engine comprises a plurality of variational autoencoders (VAEs), wherein each VAE of the plurality of VAEs is trained on a respective variability source of a plurality of variability sources; and deconvoluting, by the domain adaptation engine, the cfDNA fragmentation data using the ATAC-Seq data to generate the deconvoluted cfDNA fragmentation data comprises: encoding, by the plurality of VAEs, the cfDNA fragmentation data into a plurality of latent space representations; concatenating, by the domain adaptation engine, the latent space representations from each VAE of the plurality of VAEs to form a combined latent space representation; and processing, by the domain adaptation engine, the combined latent space representation to generate the deconvoluted cfDNA fragmentation data.

Example 14 is a non-transitory computer-readable medium storing instructions that, when executed by one or more processors, cause a computing system to: receive cell-free DNA (cfDNA) fragmentation data corresponding to a cfDNA sample from a subject; process the cfDNA fragmentation data using a pre-trained machine learning (ML) system to identify tissue-specific chromatin accessibility patterns, wherein the pre-trained ML system: (a) has been trained on single-cell Assay for Transposase-Accessible Chromatin using sequencing (ATAC-Seq) data from multiple tissue types, and (b) is configured to translate between ATAC-Seq data and cfDNA fragmentation data; detect, based on the tissue-specific chromatin accessibility patterns, a diseased tissue signature within the cfDNA fragmentation data; and generate a tissue-of-origin prediction for the diseased tissue signature based on the identified tissue-specific chromatin accessibility patterns.

Example 15 is the non-transitory computer-readable medium of any previous or subsequent Example, wherein the instructions cause the processor to further execute processor-executable instructions stored in the non-transitory computer-readable medium to: generate a confidence score associated with the tissue-of-origin prediction; and output the confidence score along with the tissue-of-origin prediction.

Example 16 is the non-transitory computer-readable medium of any previous or subsequent Example wherein the instructions to process the cfDNA fragmentation data using the pre-trained ML system to identify tissue-specific chromatin accessibility patterns cause the processor to further execute processor-executable instructions stored in the non-transitory computer-readable medium to: input the cfDNA fragmentation data into a plurality of variational autoencoders (VAEs), each VAE corresponding to a different variability source, wherein each VAE of the plurality of VAEs, encodes the cfDNA fragmentation data into a respective latent space representation; concatenate the latent space representations from each VAE of the plurality of VAEs to form a combined latent space representation; and identify the tissue-specific chromatin accessibility patterns from the combined latent space representation.

Example 17 is the non-transitory computer-readable medium of any previous or subsequent Example, wherein the instructions to detect, based on the tissue-specific chromatin accessibility patterns, the diseased tissue signature within the cfDNA fragmentation data cause the processor to further execute processor-executable instructions stored in the non-transitory computer-readable medium to: compare the tissue-specific chromatin accessibility patterns identified from processing the cfDNA fragmentation data by the pre-trained ML system to reference patterns derived from healthy tissue samples; and identify deviations in the tissue-specific chromatin accessibility patterns that exceed a predetermined threshold.

Example 18 is the non-transitory computer-readable medium of any previous or subsequent Example, wherein the instructions cause the processor to further execute processor-executable instructions stored in the non-transitory computer-readable medium to: generate a disease state prediction based on the diseased tissue signature and the tissue-of-origin prediction; and output the disease state prediction along with the tissue-of-origin prediction.

Example 19 is the non-transitory computer-readable medium of any previous or subsequent Example, wherein the instructions cause the processor to further execute processor-executable instructions stored in the non-transitory computer-readable medium to generate simulated cfDNA fragmentation data for training or validating the pre-trained ML system by: obtaining the ATAC-Seq data from multiple tissue types; selecting genomic regions from the ATAC-Seq data based on known cfDNA fragmentation patterns; applying a fragmentation model to the selected genomic regions to generate simulated cfDNA fragments; and combining the simulated cfDNA fragments with background noise derived from blood cell data to generate the simulated cfDNA fragmentation data.

Example 20 is a method for training a machine learning model to detect tissue-of-origin from cell-free DNA (cfDNA) samples, comprising: obtaining single-cell Assay for Transposase-Accessible Chromatin using sequencing (ATAC-Seq) data from multiple tissue types; generating simulated cfDNA fragmentation data by combining blood cell data with the ATAC-Seq data at varying proportions; training a machine learning (ML) system using the ATAC-Seq data and the simulated cfDNA fragmentation data, wherein the machine learning model is configured to: (a) learn tissue-specific chromatin accessibility patterns from the ATAC-Seq data, (b) translate between ATAC-Seq data and cfDNA fragmentation data, and (c) predict tissue-of-origin from cfDNA fragmentation data; validating the ML system as trained using a set of real cfDNA samples with known tissue-of-origin; and storing the ML system as validated for subsequent use in detecting tissue-of-origin from cfDNA samples.

Example 21 is the method of any previous or subsequent Example, wherein generating simulated cfDNA fragmentation data comprises: selecting a subset of genomic regions from the ATAC-Seq data based on known cfDNA fragmentation patterns; applying a fragmentation model to the subset of genomic regions to generate simulated cfDNA fragments; and combining the simulated cfDNA fragments with background noise derived from the blood cell data to generate the simulated cfDNA fragmentation data.

Example 22 is the method of any previous or subsequent Example, further comprising: fine-tuning the ML system using transfer learning techniques on a held-out set of real cfDNA samples with known tissue-of-origins; evaluating the ML system's performance on the held-out set of real cfDNA samples; and iteratively adjusting the ML system's hyperparameters to optimize its tissue-of-origin prediction accuracy across multiple tissue types and varying proportions of cfDNA in the samples.

Example 23 is the method of any previous or subsequent Example, wherein the ML system comprises a plurality of variational autoencoders (VAEs) and training the ML system comprises: training the plurality of VAEs in parallel, each VAE corresponding to a different variability source, wherein the variability sources comprise at least two of: a technology-specific variability source; a tissue type variability source; a blood cell-type proportion variability source; and a remaining variability source.

Example 24 is the method of any previous or subsequent Example, wherein training each VAE comprises: encoding input data into a latent space representation specific to the corresponding variability source; applying a regularization term to the latent space representation to encourage disentanglement of features; and decoding the latent space representation to reconstruct the input data.

Example 25 is the method of any previous or subsequent Example, further comprising: concatenating the latent space representations from each VAE to form a combined latent space representation; and using the combined latent space representation to predict tissue-of-origin from the cfDNA fragmentation data.

Example 26 is the method of any previous or subsequent Example, wherein the ML system, once trained, is configured to: process new cfDNA fragmentation data through each VAE as trained to generate respective latent space representations; combine the latent space representations to create a deconvoluted representation of the new cfDNA fragmentation data; and use the deconvoluted representation to predict the tissue-of-origin for the new cfDNA fragmentation data.

What is claimed is:

1. A computing apparatus comprising:
a computer-readable storage media comprising processor-executable instructions stored thereon; and
a processor coupled to the computer-readable storage media and configured to execute the processor-executable instructions that, when executed by the processor, direct the computing apparatus to at least:
receive a cell-free DNA (cfDNA) sample from a subject;
process the cfDNA sample to generate cfDNA fragmentation data;
input the cfDNA fragmentation data into a pre-trained machine learning (ML) system, wherein the pre-trained ML system:
(a) has been trained on cell-type-specific Assay for Transposase-Accessible Chromatin using sequencing (ATAC-Seq) data from multiple tissue types; and
(b) is configured to translate between ATAC-Seq data and cfDNA fragmentation data;
analyze the cfDNA fragmentation data using the pre-trained ML system to identify tissue-specific chromatin accessibility patterns;
predict a tissue-of-origin for the cfDNA sample based on the identified tissue-specific chromatin accessibility patterns; and
generate a prediction of the tissue-of-origin for the cfDNA sample.

2. The computing apparatus of claim 1, wherein:
the pre-trained ML system comprises a plurality of variational autoencoders (VAEs), each trained within a respective variability source of a plurality of variability sources; and
the processor-executable instructions to analyze the cfDNA fragmentation data using the pre-trained ML system to identify tissue-specific accessibility patterns, when executed by the processor, further direct the computing apparatus to:
encode, by the plurality of VAEs, the cfDNA fragmentation data into a plurality of latent spaces representations, wherein each VAE encodes the cfDNA fragmentation data into a respective latent space representations corresponding to a respective variability source; and
generate deconvoluted cfDNA fragmentation data based on the cfDNA fragmentation data as encoded into the plurality of latent space representations.

3. The computing apparatus of claim 2, wherein the plurality of variability sources that the plurality of VAEs are trained on comprises one or more of:
a technology-specific variability source;
a tissue type variability source;
a blood cell-type proportion variability source; and
a remaining variability source.

4. The computing apparatus of claim 1, wherein the processor-executable instructions to predict the tissue-of-origin for the cfDNA sample based on the identified tissue-specific chromatin accessibility patterns, when executed by the processor, further direct the computing apparatus to:
analyze a plurality of fragmentation patterns within the cfDNA fragmentation data as processed by the pre-trained ML system, wherein the plurality of fragmentation patterns comprises one or more of fragment size distribution patterns, end motif patterns, or breakpoint patterns;
compare the plurality of fragmentation patterns to reference patterns derived from ATAC-Seq data associated with known tissue types to identify tissue-specific chromatin accessibility signatures; and
determine the tissue-of-origin prediction based on the identified tissue-specific chromatin accessibility signatures.

5. The computing apparatus of claim 1, wherein the pre-trained ML system comprises a neural network trained on paired ATAC-Seq and cfDNA fragmentation data from known tissue types, wherein the neural network comprises:
an encoder network configured to:
receive training cfDNA fragmentation data as input; and
encode the training cfDNA fragmentation data into a latent space representation;
a decoder network configured to:
receive the latent space representation from the encoder network; and
reconstruct the ATAC-Seq data from the latent space representation;
wherein the neural network is trained to minimize the difference between:
the ATAC-Seq data as reconstructed in an output from the decoder network; and
the paired ATAC-Seq data corresponding to the training cfDNA fragmentation data submitted as an input; and
wherein the encoder network, as trained, is used to process the cfDNA fragmentation data to identify the tissue-specific chromatin accessibility patterns present within the cfDNA sample.

6. The computing apparatus of claim 1, wherein:
the pre-trained ML system comprises a plurality of variational autoencoders (VAEs), each VAE corresponding to a different variability source, wherein each VAE is configured to:
receive the cfDNA fragmentation data as input; and
encode the cfDNA fragmentation data into a respective latent space representation; and
the processor-executable instructions to analyze the cfDNA fragmentation data using the pre-trained ML system to identify the tissue-specific chromatin accessibility patterns, when executed by the processor, further direct the computing apparatus to:
concatenate the latent space representations from each VAE of the plurality of VAEs to form a combined latent space representation; and
process the combined latent space representation to identify the tissue-specific chromatin accessibility patterns.

7. The computing apparatus of claim 1, wherein the pre-trained ML system is configured to identify tissue-of-origin for at least three of:
breast cancer;
colorectal cancer;
lung cancer;
prostate cancer;
liver cancer;
kidney cancer;
stomach cancer;
acute myeloid leukemia (AML) cell-types;
autoimmune diseases; and
organ transplant rejection.

8. A computer-implemented method for predicting tissue-of-origin from cell-free DNA (cfDNA) samples, wherein the method comprises:
receiving, by a domain adaptation engine, a cell-free DNA (cfDNA) sample from a subject;
generating, by the domain adaptation engine, cfDNA fragmentation data from the cfDNA sample;
deconvoluting, by the domain adaptation engine, the cfDNA fragmentation data using Assay for Transposase-Accessible Chromatin using sequencing (ATAC-Seq) data from multiple tissue types to generate deconvoluted cfDNA fragmentation data;
detecting, by the domain adaptation engine, a diseased tissue signature within the deconvoluted cfDNA fragmentation data; and
generating, by the domain adaptation engine, a tissue-of-origin prediction for the diseased tissue signature based on the deconvoluted cfDNA fragmentation data.

9. The method of claim 8, wherein deconvoluting, by the domain adaptation engine, the cfDNA fragmentation data using ATAC-Seq data comprises:
encoding, by the domain adaptation engine, the cfDNA fragmentation data into a plurality of latent space representations, wherein each latent space representation corresponds to a respective variability source of a plurality of variability sources; and
generating, by the domain adaptation engine, the deconvoluted cfDNA fragmentation data based on the cfDNA fragmentation data as encoded into the plurality of latent space representations.

10. The method of claim 9, wherein the plurality of variability sources comprises one or more of:
a technology specific variability source;
a tissue or cell type variability source;
a blood cell-type proportion variability source; and
a remaining variability source.

11. The method of claim 8, wherein the method further comprises:
generating, by the domain adaptation engine, a confidence score associated with the tissue-of-origin prediction; and
transmitting, by the domain adaptation engine, the confidence score along with the tissue-of-origin prediction to a client device, wherein the confidence score and the tissue-of-origin prediction are displayed via a user interface of the client device.

12. The method of claim 8, wherein detecting, by the domain adaptation engine, the diseased tissue signature within the deconvoluted cfDNA fragmentation data comprises:
analyzing, by the domain adaptation engine, a plurality of fragmentation patterns within the deconvoluted cfDNA fragmentation data, wherein the fragmentation patterns comprise one or more of fragment size distribution patterns, end motif patterns, or breakpoint patterns;
comparing, by the domain adaptation engine, the plurality of fragmentation patterns to reference patterns derived from ATAC-Seq data associated with known tissue types to identify tissue-specific chromatin accessibility signatures; and
identifying, by the domain adaptation engine, the diseased tissue signature based on deviations in the identified tissue-specific chromatin accessibility signatures from expected patterns of healthy tissue.

13. The method of claim 8, wherein:
the domain adaptation engine comprises a plurality of variational autoencoders (VAEs), wherein each VAE of the plurality of VAEs is trained on a respective variability source of a plurality of variability sources; and
deconvoluting, by the domain adaptation engine, the cfDNA fragmentation data using the ATAC-Seq data to generate the deconvoluted cfDNA fragmentation data comprises:
encoding, by the plurality of VAEs, the cfDNA fragmentation data into a plurality of latent space representations;
concatenating, by the domain adaptation engine, the latent space representations from each VAE of the plurality of VAEs to form a combined latent space representation; and
processing, by the domain adaptation engine, the combined latent space representation to generate the deconvoluted cfDNA fragmentation data.

14. A non-transitory computer-readable medium storing instructions that, when executed by one or more processors, cause a computing system to:
receive cell-free DNA (cfDNA) fragmentation data corresponding to a cfDNA sample from a subject;
process the cfDNA fragmentation data using a pre-trained machine learning (ML) system to identify tissue-specific chromatin accessibility patterns, wherein the pre-trained ML system:
(a) has been trained on single-cell Assay for Transposase-Accessible Chromatin using sequencing (ATAC-Seq) data from multiple tissue types, and
(b) is configured to translate between ATAC-Seq data and cfDNA fragmentation data;
detect, based on the tissue-specific chromatin accessibility patterns, a diseased tissue signature within the cfDNA fragmentation data; and
generate a tissue-of-origin prediction for the diseased tissue signature based on the identified tissue-specific chromatin accessibility patterns.

15. The non-transitory computer-readable medium of claim 14, wherein the instructions cause the processor to further execute processor-executable instructions stored in the non-transitory computer-readable medium to:
generate a confidence score associated with the tissue-of-origin prediction; and
output the confidence score along with the tissue-of-origin prediction.

16. The non-transitory computer-readable medium of claim 14 wherein the instructions to process the cfDNA fragmentation data using the pre-trained ML system to identify tissue-specific chromatin accessibility patterns cause the processor to further execute processor-executable instructions stored in the non-transitory computer-readable medium to:
input the cfDNA fragmentation data into a plurality of variational autoencoders (VAEs), each VAE corresponding to a different variability source, wherein each VAE of the plurality of VAEs, encodes the cfDNA fragmentation data into a respective latent space representation;
concatenate the latent space representations from each VAE of the plurality of VAEs to form a combined latent space representation; and
identify the tissue-specific chromatin accessibility patterns from the combined latent space representation.

17. The non-transitory computer-readable medium of claim 14, wherein the instructions to detect, based on the tissue-specific chromatin accessibility patterns, the diseased tissue signature within the cfDNA fragmentation data cause the processor to further execute processor-executable instructions stored in the non-transitory computer-readable medium to:
compare the tissue-specific chromatin accessibility patterns identified from processing the cfDNA fragmentation data by the pre-trained ML system to reference patterns derived from healthy tissue samples; and
identify deviations in the tissue-specific chromatin accessibility patterns that exceed a predetermined threshold.

18. The non-transitory computer-readable medium of claim 14, wherein the instructions cause the processor to further execute processor-executable instructions stored in the non-transitory computer-readable medium to:
generate a disease state prediction based on the diseased tissue signature and the tissue-of-origin prediction; and
output the disease state prediction along with the tissue-of-origin prediction.

19. The non-transitory computer-readable medium of claim 14, wherein the instructions cause the processor to further execute processor-executable instructions stored in the non-transitory computer-readable medium to generate simulated cfDNA fragmentation data for training or validating the pre-trained ML system by:
obtaining the ATAC-Seq data from multiple tissue types;
selecting genomic regions from the ATAC-Seq data;
applying a fragmentation model to the selected genomic regions to generate simulated cfDNA fragments; and
combining the simulated cfDNA fragments with background noise derived from blood cell data to generate the simulated cfDNA fragmentation data.

20. A method for training a machine learning model to detect tissue-of-origin from cell-free DNA (cfDNA) samples, comprising:
obtaining single-cell Assay for Transposase-Accessible Chromatin using sequencing (ATAC-Seq) data from multiple tissue types;
generating simulated cfDNA fragmentation data by combining blood cell data with the ATAC-Seq data at varying proportions;
training a machine learning (ML) system using the ATAC-Seq data and the simulated cfDNA fragmentation data, wherein the machine learning model is configured to:
(a) learn tissue-specific chromatin accessibility patterns from the ATAC-Seq data,
(b) translate between ATAC-Seq data and cfDNA fragmentation data, and
(c) predict tissue-of-origin from cfDNA fragmentation data;
validating the ML system as trained using a set of real cfDNA samples with known tissue-of-origin; and
storing the ML system as validated for subsequent use in detecting tissue-of-origin from cfDNA samples.

21. The method of claim 20, wherein generating simulated cfDNA fragmentation data comprises:
selecting a subset of genomic regions from the ATAC-Seq data based on known cfDNA fragmentation patterns;
applying a fragmentation model to the subset of genomic regions to generate simulated cfDNA fragments; and
combining the simulated cfDNA fragments with background noise derived from the blood cell data to generate the simulated cfDNA fragmentation data.

22. The method of claim 20, further comprising:
fine-tuning the ML system using transfer learning techniques on a held-out set of real cfDNA samples with known tissue-of-origins;
evaluating the ML system's performance on the held-out set of real cfDNA samples; and
iteratively adjusting the ML system's hyperparameters to optimize its tissue-of-origin prediction accuracy across multiple tissue types and varying proportions of cfDNA in the samples.

23. The method of claim 20, wherein the ML system comprises a plurality of variational autoencoders (VAEs) and training the ML system comprises:
training the plurality of VAEs in parallel, each VAE corresponding to a different variability source, wherein the variability sources comprise at least two of:
a technology-specific variability source;
a tissue type variability source;
a blood cell-type proportion variability source; and
a remaining variability source.

24. The method of claim 23, wherein training each VAE comprises:
encoding input data into a latent space representation specific to the corresponding variability source;
applying a regularization term to the latent space representation to encourage disentanglement of features; and
decoding the latent space representation to reconstruct the input data.

25. The method of claim 24, further comprising:
concatenating the latent space representations from each VAE to form a combined latent space representation; and
using the combined latent space representation to predict tissue-of-origin from the cfDNA fragmentation data.

26. The method of claim 23, wherein the ML system, once trained, is configured to:
process new cfDNA fragmentation data through each VAE as trained to generate respective latent space representations;
combine the latent space representations to create a deconvoluted representation of the new cfDNA fragmentation data; and
use the deconvoluted representation to predict the tissue-of-origin for the new cfDNA fragmentation data.
